(19) United States
(12) Patent Application Publication
(10) Pub. No.: US 2026/0210798 A1
(43) Pub. Date: Jul. 23, 2026

(54) SYSTEM AND METHOD FOR GENERATING REVERSE-FLOW TSUNAMI WAVES UNDER LABORATORY CONDITIONS

(71) Applicant: UNIVERSIDAD CATÓLICA DE LA SANTÍSIMA CONCEPCIÓN, Concepción (CL)

(72) Inventor: Rafael Enrique ARÁNGUIZ MUÑOZ, Concepción (CL)

(21) Appl. No.: 18/877,638

(22) PCT Filed: Jun. 24, 2022
(86) PCT No.: PCT/CL2022/050064

§ 371 Date: Oct. 1, 2025

(51) Int. Cl. G01M 10/00

(52) U.S. Cl. CPC G01M 10/00

(57) ABSTRACT

A system and method for generating tsunami waves in reversible flow under laboratory conditions, wherein the system comprises: an elongated simulation channel having first and second side regions and a measurement region positioned between the first and second side regions; first and second basal gates configured to selectively control first and second fluid outlets from the elongated channel by opening and closing them and first and second pop-up gates positioned in the first and second side regions, respectively, at least one fluid driving pump, flow measuring and control elements, a fluid circulation line comprising a main fluid line, a first fluid inlet line in the first side region and a second fluid inlet line in the second side region; and a control system.

### DESCRIPTION

### Technical Field of the Invention

The present invention relates to the field of equipment, apparatus, and/or procedures for hydrodynamic tests or trials, and in particular provides a system and method for generating reverse-flow tsunami waves under laboratory conditions.

### Background of the Invention

A tsunami is a complex phenomenon, and reproducing its behavior in the laboratory has been challenging. The systems currently available in the state of the art can simulate only a wave in one direction, without the return flow, or generate a wave with a short period that does not correspond to a full-scale tsunami wave.

For example, JPH07120352A describes a water tank for simulating circular streams, comprising a pair of reverse rotation impellers disposed in a circular passage of the water tank, which circulates water upward and downward, respectively, within the water tank. On the other hand, CN101561345 A provides a bidirectional experimental water tank for mud and sand hydraulics and dynamics. The water tank comprises a water tank, where two ends of the water tank are provided with symmetrical water outlets, transition sections communicated to the water outlets; a main pipe disposed between the transition sections; a bidirectional axial flow water pump, an electric regulating valve, and a bidirectional electromagnetic flowmeter alternately disposed in the main pipe; and a motor driving the water pump controlled by a variable frequency actuator.

However, the state of the art is deficient in providing a system that allows reproducing both the flood and the return flow stage, respecting both the time scale of the actual phenomenon and the flow depth and speed conditions. Consequently, a system and method are needed to address the shortcomings in the current state of the art.

### SUMMARY OF THE INVENTION

The present invention provides a system for generating reverse-flow tsunami waves under laboratory conditions characterized in that it comprises: an elongated simulation channel (1) having a first side region, a second side region in a position opposite to the first side region and a measurement region positioned between the first side region and the second side region; a first basal gate (9) configured to selectively control a first fluid outlet from the elongated channel (1) by opening and closing it and a first emerging gate (10) positioned in the first side region; a second basal gate (11) configured to selectively control a second fluid outlet from the elongated channel (1) by opening and closing it and a second emerging gate (12) positioned in the second side region, at least one fluid driving pump (2), flow measuring and control means (4, 6, 7), a fluid circulation line comprising a main fluid line (5), a first fluid inlet line (8) in the first side region and a second fluid inlet line (15) in the second side region; and a control system (17, 18) of the at least one driving pump (2), of the first basal gate (9), of the second basal gate (11), of the first emerging gate (10), of the second emerging gate (12), and of the flow measuring and control means (4, 6, 7).

In a second object of the present invention, there is provided a method for generating tsunami waves with reversible flow under laboratory conditions, characterized by comprising the steps of:
provide a system comprising: an elongated simulation channel (1) having a first side region, a second side region in a position opposite to the first side region and a measurement region positioned between the first side region and second side region; a first basal gate (9) configured to selectively control a first fluid outlet from the elongated channel (1) by opening and closing it and a first emerging gate (10) positioned in the first side region; a second basal gate (11) configured to selectively control a second fluid outlet from the elongated channel (1) by opening and closing it and a second emerging gate (12) positioned in the second side region, at least one fluid driving pump (2), flow measuring and control means (4, 6, 7), a fluid circulation line comprising a main fluid line (5), a first fluid inlet line (8) in the first side region and a second fluid inlet line (15) in the second side region; and a control system (17, 18) of the at least one driving pump (2), of the first basal gate (9), of the second basal gate (11), of the first emerging gate (10), of the second emerging gate (12), and of the flow measuring and control means (4, 6, 7);
performing a first experiment of generating a tsunami wave in a first direction by means of a flow driven by the at least one fluid driving pump (2) through the main line (5), and the first fluid inlet line (8), and by controlling the fluid level by means of the second emerging gate (12), the second basal gate (11), and the control system (17, 18); and
performing a second experiment of generating a tsunami wave in a second direction by means of a flow driven by the at least one fluid driving pump (2) through the main flow line (5), and the second fluid inlet line (15), and by controlling the fluid level by means of the first emerging gate (10), the first basal gate (9), and the control system (17, 18).

### BRIEF DESCRIPTION OF THE DRAWINGS

FIG. 1 illustrates a schematic side view of a first embodiment of the system, which is the subject matter of the present invention.

### DETAILED DESCRIPTION OF THE INVENTION

In the following, the present invention will be described in detail, referring to the accompanying figures of the present application.

In a first object of the present invention, there is provided a system for generating tsunami waves in a reversible flow under laboratory conditions, essentially comprising:
an elongated simulation channel (1) having a first side region, a second side region in a position opposite to the first side region, and a measurement region positioned between the first side region and the second side region;
a first fluid outlet, a first basal gate (9) configured to open and close the first fluid outlet, and a first emerging gate (10) positioned in the first side region;
a second fluid outlet, a second basal gate (11) configured for opening and closing the second fluid outlet, and a second emerging gate (12) positioned in the second side region;
at least one fluid driving pump (2);
flow measuring and control means (4, 6, 7);
a first fluid inlet line (8) configured to inject fluid in the first side region;
a second fluid inlet line (15) configured to inject fluid in the second side region; and
a control system (17, 18) of the at least one driving pump (2), of the first basal gate (9), of the second basal gate (11), of the first emerging gate (10), of the second emerging gate (12), and of the flow measuring and control means (4, 6, 7).

In the context of the present application, without limiting the scope thereof, the expression “at least one” shall be understood as one or more of the elements referred to. The number of elements referred to by the expression “at least one” does not limit the scope of this application. Additionally, when more than one element referred to in the expression “at least one” is provided, said elements may or may not be identical to each other without limiting the scope of the present application.

In the context of the present invention, not limiting the scope thereof, it will be understood that the simulation channel (1) has an elongated shape when its length in one direction, which will be understood as an elongated direction, is greater than its length in either of the two directions perpendicular to the elongated direction. In this regard, for example, and without limiting the scope of the present invention, the length in the elongated direction may be greater than 5-fold the length in either of the two perpendicular directions, preferably greater than 10-fold the length in either of the two perpendicular directions, and even more preferably greater than 20-fold the length in either of the two perpendicular directions.

Additionally, without limiting the scope of the present invention, it will be understood that the first side region and the second side region are laterally with respect to the elongated direction of the elongated channel (1). The extension of the first side region and the second side region along the elongated direction of the elongated channel (1) does not limit the scope of the present invention as long as it allows obtaining a simulation region positioned between each other. Additionally, the first side region and the second side region may or may not have the same extension without limiting the scope of the protection requested. In a preferred embodiment, without limiting the scope of the present invention, the first side region and the second side region have the same extension.

The elongated channel (1) that forms part of the system that is the present invention subject matter, further comprises a simulation region positioned between the first side region and the second side region. The extension of said simulation region along the elongated direction of the elongated channel (1) does not limit the scope of the present invention, and it will depend, for example, on the extensions of the first side region and the second side region, as well as on the length of the elongated channel (1) along the elongated direction.

In said simulation region, preferably and without limiting the scope of the present invention, one or more elements that allow obtaining measurements when performing the simulation of a particular phenomenon may be positioned. For example, and without limiting the scope of the present invention, in said region of simulation may be placed at least one fluid level sensor, at least one vertical fluid speed sensor, at least one transverse fluid speed sensor, at least one video camera, at least one image capture camera, at least one light sources, at least one fluid pressure sensor, at least one fluid temperature sensor, as well as a combination thereof.

In a preferred embodiment, without limiting the scope of the present invention, the measurement region may comprise a change in depth that may be an indentation, which has a greater depth than the first side region and the second side region, or an elevation, which has a smaller depth. In this depth change area, installation of elements for testing and additional elements for measuring hydrodynamic variables that allow the simulation of a particular phenomenon can be allowed. For example, in a preferred embodiment, without limiting the scope of the present invention, it is possible to position scale coastal structures, scale buildings, beaches with a smooth slope and fixed material, beaches with a smooth slope and removable granular material, scale mitigation forests, power generation systems, pressure measurement instruments, or an immersion pump in said indentation. In this exemplary embodiment, in addition to and without limiting the scope of the present invention, the system may comprise a drain line that fluidly connects said immersion pump with a fluid storage tank.

The system that is the present invention subject matter comprises a first basal gate (9) configured to selectively control a first fluid outlet from the elongated channel (1) by opening and closing it, and a first emerging gate (10) positioned in the first side region of the elongated channel (1). Similarly, the system that is the object of the present invention comprises a second basal gate (11) configured to selectively control a second fluid outlet from the elongated channel (1) by opening and closing it, and a second emerging gate (12) positioned in the second side region of the elongated channel (1).

In the context of the present invention, without limiting the scope thereof, it will be understood that the basal gate (either the first basal gate (9) or the second basal gate (11)) allows the opening and closing of its corresponding fluid outlet (either the first fluid outlet or the second fluid outlet, respectively). For this, said basal gate (9, 11) can acquire at least two positions, which will be referred to as the open position and the closed position, respectively. However, in some preferred embodiments, said base gate (9, 11) can, in addition and without limiting the scope of the present invention, acquire at least one intermediate position between said open position and said closed position. The means by which said basal gate (9, 11) is allowed to transit from the open position to the closed position, or vice versa, do not limit the scope of the present invention. For example, in a preferred embodiment and without limiting the scope of the present invention, said basal gate (9, 11) can perform a substantially horizontal movement between said open position and said closed position. In another exemplary embodiment, without limiting the scope of the present invention, said basal gate (9, 11) can perform a pivoting movement between said open position and said closed position. Additionally, one or more actuators and one or more transmission elements that allow controlling the position of the basal gate (9, 11) may be provided. For example, and without limiting the scope of the present invention, motors, hydraulic arms, pneumatic arms, chains, ropes, springs, as well as a combination thereof, may be provided to control the position of the basal gate (9, 11).

It should be understood, without limiting the scope of the present invention, that the first basal gate (9) and the second basal gate (11) may or may not be identical to each other. In addition, the means that are provided to control the position of the first basal gate (9), and of the second basal gate (11) may or may not be identical to each other without limiting the scope of the present invention.

In the context of the present invention, without limiting the scope thereof, it will be understood that the emerging gate (either the first emerging gate (10) or the second emerging gate (12)) allows controlling the water level inside the measurement region. For this, said emerging gate (10, 12) can acquire at least two positions, which will be named retracted position, in which the emerging gate (10, 12) does not protrude in relation to the bottom of the corresponding side region, and deployed position in which the emerging gate (10, 12) protrudes in relation to the bottom of the corresponding side region. However, in some preferred embodiments, said emerging gate (10, 12) can, in addition and without limiting the scope of the present invention, acquire a plurality of deployed positions, each of which protrudes at a corresponding height in relation to the bottom of its corresponding side region. The means by which said emerging gate (10, 12) is allowed to transit from the retracted position to the deployed position, or vice versa, do not limit the scope of the present invention. For example, in a preferred embodiment and without limiting the scope of the present invention, said emerging gate (10, 12) can perform a substantially vertical movement between said retracted position and said deployed position. In another embodiment, without limiting the scope of the present invention, said emerging gate (9, 11) is capable of performing a pivoting movement between said retracted position and said deployed position. Additionally, one or more actuators and one or more transmission elements that allow controlling the position of the emerging gate (10, 12) may be provided. For example, and without limiting the scope of the present invention, motors, hydraulic arms, pneumatic arms, chains, ropes, springs, as well as a combination thereof, may be provided to control the position of the basal gate (10, 12).

It should be understood, without limiting the scope of the present invention, that the first emerging gate (10) and the second emerging gate (12) may or may not be identical to each other. In addition, the means that are provided to control the position of the first emerging gate (10), and of the second emerging gate (12) may or may not be identical to each other without limiting the scope of the present invention.

The system that is the present invention subject matter comprises at least one fluid driving pump (2) that aims to drive the fluid into the elongated channel (1). Any number or type of pump may be used without limiting the scope of the present invention. If more than one pump is provided, said fluid driving pumps (2) could work in series or in parallel without limiting the scope of the present invention. In a more preferred embodiment, without limiting the scope of the present invention, said at least one driving pump (2) may comprise a corresponding frequency converter, in order to control the flow rate of the fluid that is driven by said at least one driving pump (2).

The system that is the present invention subject matter further comprises flow measuring and control means (4, 6, 7), which have the purpose of controlling the flow rate and direction of the fluid circulating through the main fluid line (5), and the first and second side inlet lines (8, 15). In this regard, said flow measuring and control means (4, 6, 7) may comprise, without being limited thereto, valves, flow sensors, diverters, stopcocks, as well as a combination thereof. In a preferred embodiment, without limiting the scope of the present invention, the flow measurement and control means (4, 6, 7) may comprise at least one flowmeter (4).

The system that is subject matter of the present invention further comprises a control system (17, 18) of the fluid driving pumps (2), of the flow measuring and control means (4, 6, 7), of the first basal gate (9), of the second basal gate (11), of the first emerging gate (10), and of the second emerging gate (12). As previously indicated, said control system (17, 18) may include, without limiting the scope of the present invention, motors, electronic elements, hydraulic arms, pneumatic arms, chains, ropes, springs, as well as a combination thereof. Additionally, said control system (17, 18) can be either manual or automatic without limiting the scope of the present invention. In a preferred embodiment, without limiting the scope of the present invention, the system may include a computer or processor (18) configured or programmed to control the at least one fluid driving pump (2), of the flow measuring and control means (4, 6, 7), of the first basal gate (9), of the second basal gate (11), of the first emerging gate (10), and of the second emerging gate (12). For this, for example and without limiting the scope of the present invention, said computer or processor (18) may include one or more interfaces, both physical and logical, that allow it to interact to the first basal gate (9), the second basal gate (11), the first emerging gate (10), and the second emerging gate (12). In a more preferred embodiment, the system includes an electrical connection board (17) that is operatively connected to the computer or processor (18), and said computer or processor (18) is configured to control the energization of the different components of the system by said electrical connection board (17).

In another preferred embodiment, without limiting the scope of the present invention, the computer or processor (18) may be further configured to control the at least one fluid driving pump (2), and the flow measuring and control means (4, 6, 7) that are operatively connected to the main fluid line (5), and control whether the flow direction follows the first fluid inlet line (8) or the second fluid inlet line (15). In this way, for example and without limiting the scope of the present invention, the computer or processor (18) can control both the position of the first basal gate (9), the first emerging gate (10), the second basal gate (11), and the second emerging gate (12), as well as the flow rate of fluid circulating through the main fluid line (5), and whether the flow follows the first inlet line (8) or the second inlet line (15).

In a preferred embodiment, without limiting the scope of the present invention, the system can comprise at least one fluid level sensor positioned in the elongated channel (1). For example, and without limiting the scope of the present invention, the system may comprise a first fluid level sensor positioned in the first side region, a second fluid level sensor (16) positioned in the second side region, and a third fluid level sensor (13) positioned in the measurement region. If provided, any option known to a person of ordinary skill in the art may be used as a fluid level sensor without limiting the scope of the present invention. In an exemplary embodiment, without limiting the scope of the present invention, when a computer or processor (18) is provided, said computer or processor (18) may be operatively connected to the at least one fluid level sensor. In this way, the computer or processor (18) can acquire at least one measurement from the at least one fluid level sensor and use said information to control the position of the first basal gate (9), the second basal gate (11), the first emerging gate (10), and the second emerging gate (12), and/or the direction and flow rate of the fluid circulating through the main fluid line (5), and through the first fluid inlet line (8) or the second fluid inlet line (15). On the other hand, the way in which said first fluid inlet line (8) and second fluid inlet line (15) inject the fluid into the first side region and the second side region, respectively, does not limit the scope of the present invention. For example, and without limiting the scope of the present invention, said first fluid inlet line (8) and second fluid inlet line (15) may each comprise ducts entering the elongated channel (1) for the injection of fluid. However, in other preferred embodiments and without limiting the scope of the present invention, the elongated channel (1) may comprise connection portions, for example a threaded connection, to which said first fluid inlet line (8) or second fluid inlet line (15) for the injection of the fluid in the first side region or in the second side region, respectively, are connected.

In a preferred embodiment, without limiting the scope of the present invention, the system may comprise a fluid storage tank (3) downstream of the first basal gate (9) and of the second basal gate (11). However, in other preferred embodiments, without limiting the scope of the present invention, the system may comprise a first fluid storage tank positioned downstream of the first basal gate (9), and a second fluid storage tank positioned downstream of the second basal gate (11). In a more preferred embodiment, without limiting the scope of the present invention, it is possible to provide a fluid communication line between the first storage tank and the second storage tank, and a valve that selectively connects the first storage tank to the second storage tank.

In a second object of the present invention, there is provided a method for generating tsunami waves in a reversible flow under laboratory conditions, essentially comprising the steps of:
providing a system comprising: an elongated simulation channel (1) having a first side region, a second side region in a position opposite to the first side region and a measurement region positioned between the first side region and the second side region; a first basal gate (9) configured to selectively control a first fluid outlet from the elongated channel (1) by opening and closing it and a first emerging gate (10) positioned in the first side region; a second basal gate (11) configured to selectively control a second fluid outlet from the elongated channel (1) by opening and closing it and a second emerging gate (12) positioned in the second side region, at least one fluid driving pump (2), flow measuring and control means (4, 6, 7), a fluid circulation line comprising a main fluid line (5), a first fluid inlet line (8) in the first side region and a second fluid inlet line (15) in the second side region; and a control system (17, 18) of the at least one driving pump (2), of the first basal gate (9), of the second basal gate (11), of the first emerging gate (10), of the second emerging gate (12), and of the flow measuring and control means (4, 6, 7);
performing a first experiment of generating a tsunami wave in a first direction by means of a flow driven by the at least one fluid driving pump (2) through the main line (5), and the first fluid inlet line (8), and by controlling the fluid level by means of the second emerging gate (12), the second basal gate (11), and the control system (17, 18); and
performing a second experiment of generating a tsunami wave in a second direction by means of a flow driven by the at least one fluid driving pump (2) through the main flow line (5), and the second fluid inlet line (15), and by controlling the fluid level by means of the first emerging gate (10), the first basal gate (9), and the control system (17, 18).

The manner in which the first experiment and the second experiment are carried out does not limit the scope of the present invention, as long as they are carried out in a first direction and in a second direction. In this regard, for example, and without limiting the scope of the present invention, the parameters of the first experiment and the second experiment may or may not be equal to each other.

In a preferred embodiment, without limiting the scope of the present invention, prior to performing the first experiment, the method may comprise the steps of:
bringing the first basal gate (9) to a closed position, the first emerging gate (10) to a retracted position, the second basal gate (11) to an open position, and the second emerging gate (12) to a deployed position by means of said control system (17, 18); and
injecting a fluid into the first side region of the simulation channel (1) through the main fluid line (5), and the first fluid inlet line (8) until the fluid level reaches a level defined by the second emerging gate (12) by means of said control system (17, 18).

In another preferred embodiment, after performing the first experiment and prior to performing the second experiment, the method may comprise the steps of:
stop the fluid from entering the first side region of the simulation channel (1);
bringing the second basal gate (11) to a closed position, by means of the control means;
injecting fluid into the second side region of the channel through the second fluid inlet line (15) until the fluid level in the second side region reaches the fluid level in the central measurement region;
stop the fluid from entering the second side region of the channel, by means of the control system (17, 18), once the fluid level in the second side region reaches the fluid level in the first side region;
bringing the first emerging gate (10) to a deployed position and the second emerging gate (12) to a retracted position, by means of the control system (17, 18); and
opening the first basal gate (9) after the first emerging gate (10) reaches the deployed position, by means of the control system (17, 18) to empty the volume in the first side region.

According to the previously detailed description, it is possible to obtain a system and method that allows overcoming the shortcomings in the state of the art.

It should be understood that the different options described for the technical characteristics of the system and/or the method can be combined with each other, or with other alternatives known to a person of ordinary skill in the art, without limiting the scope of the protection requested.

In the following, examples of application of the system and method that are the subject of this application will be provided. The examples mentioned above are provided for a better understanding of the technology, but in no case should they be interpreted as limiting the scope of the protection requested. Additionally, details of technical characteristics described in different examples may be combined with each other, or with other options previously described or known to a person of ordinary skill in the art, in any way, provided that this does not limit the scope of protection.

### Example 1: Implementation of a Tsunami Wave Generation System at Laboratory Scale

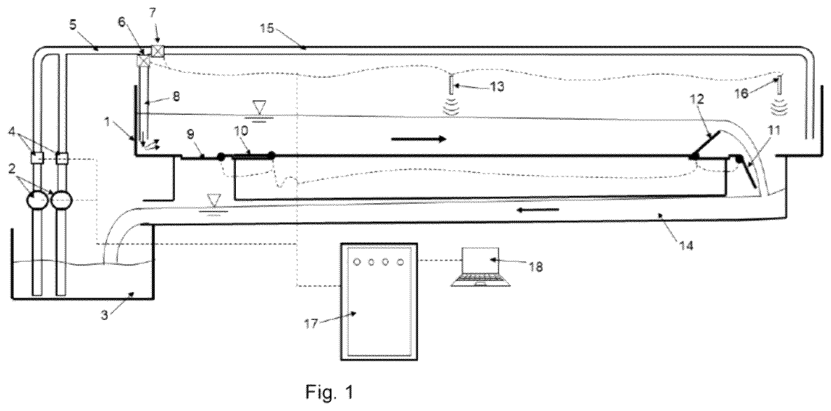

A system for generating reverse-flow tsunami waves was built, as schematically illustrated in FIG. 1, which features an elongated channel (1) measuring 20 m in length. The system has a set of centrifugal pumps (2) that suck water from an underground storage pond (3), and drive water through a pipe (5) to the elongated channel (1). In addition, a set of electromagnetic flowmeters (4) for flow control and a set of valves (6, 7) for flow direction control are provided.

To perform an experiment in one direction, from left to right in FIG. 1, a first valve (6) is opened and a second valve (7) is closed, so that the flow enters the elongated channel (1) from the left (8). The left basal gate (9) remains closed, and the left emerging gate (10) remains in retracted position. On the other hand, the right basal gate (11) is entirely open, and the right emerging gate (12) gradually rises to control the height of the flow, which is measured with a height sensor (13).

The excess of flow passing the emerging gate (12) falls into a pipe (14), leading the flow to the underground storage pond (3).

Once the experiment has ended in one direction, the flow is stopped, the right basal gate (11) is closed, the first valve (6) is closed, and the second valve (7) is opened. In addition, the pumps (2) drive the flow through the right pipe (15) in order to feed the elongated channel (1) from the right side, measuring the water level by means of the right height sensor (16) until reaching the existing water level. Once the existing volume has been reached, the pumps are stopped.

At the same time, the left emerging gate (10) rises slowly until reaching the desired level, and subsequently, the right emerging gate (12) descends slowly to the base of the channel. Then, the left basal gate (9) is opened to empty the volume of water located to the left of the left emerging gate (10). In this condition, the flow begins in the opposite direction and thus the pumps (2) drive the flow through the right pipe (15), where the height is controlled by the left gate (10), falling in a controlled manner and the excess water falls into the pipe (14), and is led to the storage pond (3).

All components of the system are controlled by the electrical panel (17) and the computer (18), which utilize software specifically developed for this system.

1. A system for generating reverse-flow tsunami waves under laboratory conditions comprising:
an elongated simulation channel having a first side region, a second side region in a position opposite to the first side region, and a measurement region positioned between the first side region and the second side region;
a first fluid outlet, a first basal gate configured to open and close the first fluid outlet, and a first emerging gate positioned in the first side region;
a second fluid outlet, a second basal gate configured for opening and closing the second fluid outlet, and a second emerging gate positioned in the second side region;
at least one fluid driving pump;
flow measuring and control means;
a first fluid inlet line configured to inject fluid in the first side region;
a second fluid inlet line configured to inject fluid in the second side region;
a control system of the at least one driving pump of the first basal gate, of the second basal gate of the first emerging gate, of the second emerging gate and the flow measuring and control means; and
a first fluid level sensor positioned in the first side region, a second fluid level sensor positioned in the second side region, and a third fluid level sensor positioned in the measurement region.

2. The system of claim 1, further comprising a fluid storage tank downstream of the first basal gate and the second basal gate.

3. The system of claim 1, comprising a first fluid storage tank positioned downstream the first basal gate, and a second fluid storage tank positioned downstream the second basal gate.

4. The system of claim 3, further comprising a fluid communication line between the first storage tank and the second storage tank and a valve selectively connecting the first storage tank to the second storage tank.

5. The system of claim 1, wherein the fluid measurement and control means comprising of at least one flowmeter.

6. The system of claim 1, wherein said at least one pump further comprises a frequency converter.

7. The system of claim 1, wherein the first basal gate, the second basal gate the first emerging gate and the second emerging gate are pivoting gates.

8. The system of claim 1, wherein the first emerging gate and the second emerging gate are configured to travel in a vertical direction.

9. The system of claim 1, wherein the measurement region comprising an indentation having a depth greater than the first side region and the second side region.

10. The system of claim 9, further comprising an immersion pump positioned in said indentation and a drain line fluidly connecting said immersion pump to a fluid storage tank.

11. The system of claim 1, further comprising a fluid speed sensor positioned in the measurement region of the channel.

12. A method for generating reverse-flow tsunami waves under laboratory conditions comprising the steps of:
providing a system comprising: an elongated simulation channel having a first side region, a second side region in a position opposite to the first side region and a measurement region positioned between the first side region and the second side region; a first basal gate configured to selectively control a first fluid outlet from the elongated channel by opening and closing it and a first emerging gate positioned in the first side region; a second basal gate configured to selectively control a second fluid outlet from the elongated channel by opening and closing it and a second emerging gate positioned in the second side region, at least one fluid driving pump, flow measuring and control means, a fluid circulation line comprising a main fluid line, a first fluid inlet line in the first side region and a second fluid inlet line in the second side region; and a control system of the at least one driving pump, of the first basal gate, of the second basal gate of the first emerging gate, of the second emerging gate, and of the measuring means, wherein the measuring means comprise a first fluid level sensor positioned in the first side region, a second fluid level sensor positioned in the second side region, and a third fluid level sensor positioned in the flow measuring and control region;
performing a first experiment of generating a tsunami wave in a first direction by means of a flow driven by the at least one fluid driving pump through the main line, and the first fluid inlet line, and by controlling the fluid level by means of the second emerging gate the second basal gate and the control system; and
performing a second experiment of generating a tsunami wave in a second direction by means of a flow driven by the at least one fluid driving pump through the main flow line, and the second fluid inlet line, and by controlling the fluid level by means of the first emerging gate, the first basal gate, and the control system.

13. The method of claim 12, wherein prior to performing the first experiment, the following steps are carried out of:
bringing the first basal gate to a closed position, the first emerging gate to a retracted position, the second basal gate to an open position, and the second emerging gate to a deployed position by means of said control system; and
injecting a fluid into the first side region of the simulation channel through the main fluid line and the first fluid inlet line until the fluid level reaches a level defined by the second emerging gate by means of said control system.

14. The method of claim 13, wherein after performing the first experiment and prior to performing the second experiment, the following steps are carried out:
bringing the second basal gate to a closed position, by means of the control means;
injecting fluid into the second side region of the channel through the second fluid inlet line until the fluid level in the second side region reaches the fluid level in the central measurement region;
stop the fluid from entering the second side region of the channel, by means of the control system, once the fluid level in the second side region reaches the fluid level in the first side region;
bringing the first emerging gate to a deployed position and the second emerging gate to a retracted position, by means of the control system; and
opening the first basal gate after the first emerging gate reaches the deployed position, by means of the control system to empty the volume in the first side region.
